(19) United States
(12) Patent Application Publication
(10) Pub. No.: US 2022/0101986 A1
(43) Pub. Date: Mar. 31, 2022

(54) METHOD FOR ADAPTIVE TRANSPORTATION SERVICES SCHEDULING FOR HEALTHCARE COST REDUCTION

(71) Applicant: KONINKLIJKE PHILIPS N.V., EINDHOVEN (NL)

(72) Inventors: Jin LIU, MALDEN, MA (US); Eran SIMHON, BOSTON, MA (US); Reza SHARIFI SEDEH, MALDEN, MA (US)

(21) Appl. No.: 17/298,043

(22) PCT Filed: Dec. 11, 2019
(86) PCT No.: PCT/EP2019/084760

§ 371 Date: May 28, 2021

Related U.S. Application Data
(60) Provisional application No. 62/779,658, filed on Dec. 14, 2018.

(51) Int. Cl. G16H 40/20; G06Q 10/06

(52) U.S. Cl. CPC G16H 40/20; G06Q 10/0635

(57) ABSTRACT

A method for scheduling patients for medical appointments, including: predicting the no-show risk and no-show cost for the patients; forecasting the cost of a transportation assistance service for the patients; optimizing the scheduling of patients based upon cost of the transportation assistance service, the no-show risk, and the no-show cost; selecting a population of patients to receive the transportation assistance service; and scheduling the population of patients for their medical appointment and transportation assistance service.

### TECHNICAL FIELD

Various exemplary embodiments disclosed herein relate generally to a method for adaptive transportation services scheduling for healthcare cost reduction.

### BACKGROUND

Patients missing medical appointments, i.e., the patient's no-show problem is very common in many healthcare settings with various no-show rates reported. The patient no-show problem may be the result of various factors, among which difficulty with transportation is an important one. It was reported that about 3.6 million Americans miss medical appointments due to transportation difficulties each year. The annual cost of missed primary care appointments is about $150 billion. Transportation assistance service has been provided by some hospitals to assist patients to attend their medical appointments. In a previous study, a 7% reduced ambulance usage per capita was found with the introduction of the traditional Uber service to a city.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for scheduling patients for medical appointments, including: predicting the no-show risk and no-show cost for the patients; forecasting the cost of a transportation assistance service for the patients; optimizing the scheduling of patients based upon cost of the transportation assistance service, the no-show risk, and the no-show cost; selecting a population of patients to receive the transportation assistance service; and scheduling the population of patients for their medical appointment and transportation assistance service.

Various embodiments are described, wherein predicting the no-show risk and no-show cost for the patients includes training no-show risk model that predicts the no-show risk.

Various embodiments are described, wherein predicting the no-show risk for a patient includes calculating the no-show risk for the patients based upon their transportation availability and calculating the no-show risk for the patients when the transportation assistance service is provided.

Various embodiments are described, wherein predicting the no-show risk and no-show cost for the patients includes determining a healthcare cost function based upon the no-show risk of the patients and the cost of a missed medical appointment.

Various embodiments are described, wherein the cost of the missed medical appointment is based upon at least one of idle time of medical providers, medical equipment, medical facilities, and decreased health outcome due to the missed appointment.

Various embodiments are described, wherein training no-show risk model that predicts the no-show risk includes using least absolute shrinkage and selection operator (LASSO) regression or random forest.

Various embodiments are described, wherein predicting the no-show risk and no-show cost for the patients includes at least one of the following data: socioeconomic factors; income; employment; insurance coverage; age; gender; social support; vehicle availability; public transport availability; previous appointment records; electronic medical records; prior appointment attendance; and prior cost records.

Various embodiments are described, wherein forecasting the cost of a transportation assistance service for the patients includes producing a model of the cost of the transportation assistance service based upon a patient address, a medical appointment location, and time of service.

Various embodiments are described, wherein forecasting the cost of a transportation assistance service for the patients includes collecting available times for appointments for the patients.

Various embodiments are described, wherein forecasting the cost of a transportation assistance service for the patients includes using the model of the cost of the transportation assistance service with available patient times as inputs.

Various embodiments are described, wherein optimizing the scheduling of patients includes determining the total healthcare cost difference with and without transportation assistance for the patients based upon no-show cost for the patients, the cost of the transportation assistance services for the patients, and a risk threshold value.

Various embodiments are described, wherein optimizing the scheduling of patients includes determining the risk threshold value that produces the lowest total healthcare cost difference with and without transportation assistance for the patients.

Various embodiments are described, wherein selecting a population of patients to receive the transportation assistance service is based upon the determined risk threshold value.

Various embodiments are described, wherein model of the cost of the transportation assistance service is one of a recurrent neural network, a long short-term memory (LSTM) recurrent neural network, a gated recurrent unit (GRU) neural network, and a time series analysis model.

Further various embodiments relate to a non-transitory machine-readable storage medium encoded with instructions for scheduling patients for medical appointments, including: instructions for predicting the no-show risk and no-show cost for the patients; instructions for forecasting the cost of a transportation assistance service for the patients; instructions for optimizing the scheduling of patients based upon cost of the transportation assistance service, the no-show risk, and the no-show cost; instructions for selecting a population of patients to receive the transportation assistance service; and instructions for scheduling the population of patients for their medical appointment and transportation assistance service.

Various embodiments are described, wherein instructions for predicting the no-show risk and no-show cost for the patients includes instructions for training no-show risk model that predicts the no-show risk.

Various embodiments are described, wherein instructions for predicting the no-show risk for a patient includes instructions for calculating the no-show risk for the patients based upon their transportation availability and instructions for calculating the no-show risk for the patients when the transportation assistance service is provided.

Various embodiments are described, wherein instructions for predicting the no-show risk and no-show cost for the patients includes instructions for determining a healthcare cost function based upon the no-show risk of the patients and the cost of a missed medical appointment.

Various embodiments are described, wherein the cost of the missed medical appointment is based upon at least one of idle time of medical providers, medical equipment, medical facilities, and decreased health outcome due to the missed appointment.

Various embodiments are described, wherein instructions for training no-show risk model that predicts the no-show risk includes using least absolute shrinkage and selection operator (LASSO) regression or random forest.

Various embodiments are described, wherein instructions for predicting the no-show risk and no-show cost for the patients includes at least one of the following data: socioeconomic factors; income; employment; insurance coverage; age; gender; social support; vehicle availability; public transport availability; previous appointment records; electronic medical records; prior appointment attendance; and prior cost records.

Various embodiments are described, wherein instructions for forecasting the cost of a transportation assistance service for the patients includes instructions for producing a model of the cost of the transportation assistance service based upon a patient address, a medical appointment location, and time of service.

Various embodiments are described, wherein instructions for forecasting the cost of a transportation assistance service for the patients includes instructions for collecting available times for appointments for the patients.

Various embodiments are described, wherein instructions for forecasting the cost of a transportation assistance service for the patients includes using the model of the cost of the transportation assistance service with available patient times as inputs.

Various embodiments are described, wherein instructions for optimizing the scheduling of patients includes instructions for determining the total healthcare cost difference with and without transportation assistance for the patients based upon no-show cost for the patients, the cost of the transportation assistance services for the patients, and a risk threshold value.

Various embodiments are described, wherein instructions for optimizing the scheduling of patients includes instructions for determining the risk threshold value that produces the lowest total healthcare cost difference with and without transportation assistance for the patients.

Various embodiments are described, wherein instructions for selecting a population of patients to receive the transportation assistance service is based upon the determined risk threshold value.

Various embodiments are described, wherein model of the cost of the transportation assistance service is one of a recurrent neural network, a long short-term memory (LSTM) recurrent neural network, a gated recurrent unit (GRU) neural network, and a time series analysis model.

### BRIEF DESCRIPTION OF THE DRAWINGS

In order to better understand various exemplary embodiments, reference is made to the accompanying drawings, wherein:

FIG. 1 illustrates a block diagram of a patient scheduling system;

FIG. 2 illustrates a method carried out by the patient scheduling system to schedule patients with a high no-show risk;

FIG. 3 illustrates a plot of the cost of transportation from an address aᵢ to a hospital where a patient has a medical appointment;

FIG. 4 illustrates a heat map 405 showing the cost of transportation services across the mapped area; and

FIG. 5 illustrates an example of scheduling optimization module finding the optimal r₀ and list of tᵢ which produces a minimum overall healthcare cost difference ΔC (r₀, {tᵢ}).

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Patients missing medical appointments, i.e., the patient's no-show problem is very common in many healthcare settings, causing reduced efficiency and increased health care costs. Transportation assistance services to assist patients to attend medical appointments have been adopted in hospitals to decrease the no-show rate. However, transportation assistance service is not free of charge, and its price may vary depending upon the day of the week and the time of day. How to select patients in need and adaptively deliver transportation assistance services with reduced cost in both transportation assistance service and healthcare no-show cost is important to not only ease financial burdens but also to improve clinical outcomes, because patients who miss their medical appointments have poorer clinical outcomes.

Embodiments of a patient scheduling system with population selection and adaptive scheduling using machine learning (for no-show risk prediction) and neural networks (for transportation service cost forecasting) are described herein to in order to achieve the following three goals: 1) reduce a no-show rate; 2) reduce the cost of transportation services to support those that have transportation challenges; and 3) increase healthcare service efficiency.

To achieve these goals, a machine learning model is disclosed that predicts the no-show risk of patients with and without transportation service assistance. A population having a high no-show risk due to not having transportation is selected as the beneficiaries for transportation services. In addition, varying transportation service prices/costs for the coming days/hours are forecasted using time series analysis and/or recurrent neural network methods. By combining information of varying transportation service prices at the different time and different days and patient/care manager's preferences (soft constraints) and patients' availabilities (hard constraints), an optimal appointment time may be scheduled for each patient to decrease their risk of no-show for an appointment.

Different patients have various no-show risk depending on their socioeconomic behavior factors, appointment type, and health conditions. It is important to identify and target patients who really need the transportation service to reduce their no-show risk. Furthermore, the price/cost of transportation service such as Uber varies depending upon the day of the week, time of day, location, and the demand for rides. Finding proper appointment times for patients in need that both have low cost for the transportation service and satisfying the patient's need is very important in order to achieve efficient cost reduction and improved healthcare outcome.

For population selection, a machine learning model (e.g., least absolute shrinkage and selection operator (LASSO) regression, random forest, etc.) is first developed to predict the no-show risk of patients with and without transportation service assistance. Patients with high no-show risk due to not having transportation are selected as the beneficiaries for transportation services.

In addition, varying transportation service costs for the coming days are forecasted using recurrent neural network models or other models that account for seasonal patterns. By combining information of varying transportation service prices at different times and different days and patient/care manager's preferences (soft constraints), and patients' availabilities (hard constraints), optimal appointment time for patients and population selection criteria may be optimized with minimized total cost of transportation service and healthcare cost to reduce patient no-show risk.

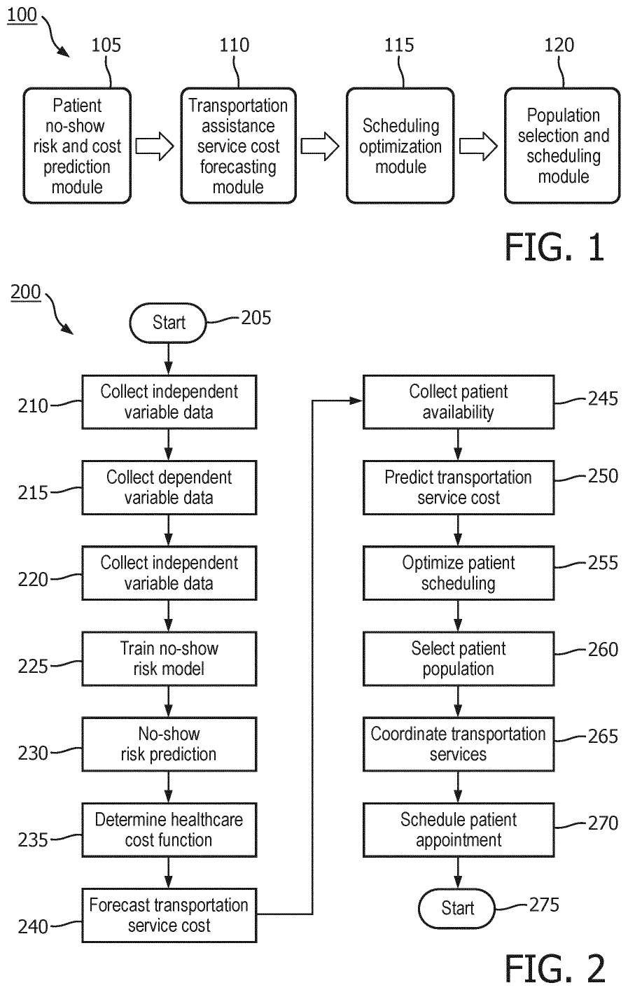

FIG. 1 illustrates a block diagram of a patient scheduling system. The patient scheduling system may include a patient no-show risk and cost prediction module 105, a transportation assistance service cost forecasting module 110, a scheduling optimization module 115, and a population selection and scheduling module 120. FIG. 2 illustrates a method carried out by the patient scheduling system to schedule patients with a high no-show risk.

The patient no-show risk and cost prediction module 105 carries out steps 210 to 235 of the method 200. First, the patient no-show risk and cost prediction module 105 collects and prepares independent variable information 210 including, for example, socioeconomic behavior factors (e.g., income, employment, insurance coverage, age, gender, social support, vehicle availability and/or public transportation), previous appointment records (time and date of appointment), and electronic medical records (EMR). Next, the patient no-show risk and cost prediction module 105 collects and prepares dependent variable information 215 from previous appointment records including whether medical appointment was attended. This information is very specific and dependent on the specific patient and their appointment history. Then the patient no-show risk and cost prediction module 105 trains a no-show risk prediction machine learning model (e.g., using LASSO regression, random forest, etc.) with variables prepared in steps 215 and 220. The patient no-show risk and cost prediction module 105 predicts the no-show risk, rᵢ, 230 for a new appointment of patient i using patient information prepared in step 210. Next, the patient no-show risk and cost prediction module 105 predicts the no-show risk, r′ᵢ, 230 for a new appointment of patient i, if the patient is provided with transportation service (for example, by setting vehicle availability and public transportation as positive). Finally, the patient no-show risk and cost prediction module 105 determines a healthcare cost function ƒ(i) 235 with no-show risk of r using previous appointment and cost records. The cost includes the revenue loss caused by facilities, equipment, and physicians idle time and potential future healthcare cost due to the patient missing their medical appointment (e.g., future emergency department visit or hospital admission cost).

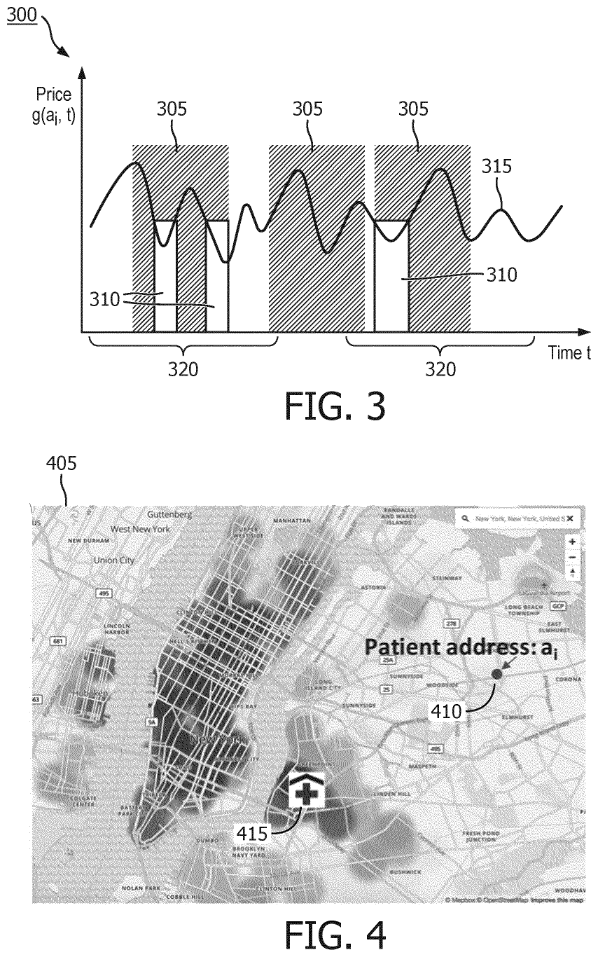

The transportation assistance service cost forecasting module 110 carries out steps 240 to 250 of the method 200. The transportation assistance service cost forecasting module 110 forecasts the transportation service cost g(a, t) 240 based on historical cost records using a recurrent neural network (e.g., long short-term memory (LSTM) or gated recurrent unit (GRU)) considering pricing variations (e.g., daily, weekly, location), where a is the address and t is the scheduled time. Other types of models may be used for modelling the transportation service cost g(a, t), e.g., time series analysis. FIG. 4 illustrates a heat map 405 showing the cost of transportation services across the mapped area. The patient address is shown 410 as well as the location of the hospital 415. This information is used by the transportation assistance service cost forecasting module 110. The transportation assistance service cost forecasting module 110 next collects available times (Tᵢ) for a new appointment for patient i through patient preferred communication methods (e.g., manual/auto phone call, email, or text massage) and patient address (aᵢ) from EMR 245. Then the transportation assistance service cost forecasting module 110 predicts transportation service cost, g(aⱼ, tᵢ) 250, for patient i scheduled at time tᵢ (tᵢ∈Tᵢ) with unit cost from step 240 and address aₜ. FIG. 3 illustrates a plot 300 of the cost of transportation from an address aᵢ to a hospital where a patient i has a medical appointment. The plot shows the patient available times 305 and a plot of the cost of the transportation service 315. Also, the times when the hospital is open are shown 320. The preferred times with reduced cost 310 are also shown. The transportation assistance service cost forecasting module 110 determines the preferred times with reduced cost 310.

The scheduling optimization module 115 carries out step 255 of the method 200. The scheduling optimization module 115 optimizes the scheduling of patients by finding an appointment time satisfying the following conditions. For patient 1, define a total healthcare cost function with assumed cut-off threshold (r₀) as independent variable as:

$$c ( {r}_{0} , i ) = \{ \begin{matrix}f ( {r}_{i}^{′} ) + g ( {a}_{i} , {t}_{i} ) , if {r}_{0} < {r}_{i} ( with transportation service ) \\ f ( {r}_{i} ) , if {r}_{0} \geq {r}_{i} ( no transportation service )\end{matrix} ,$$

where f(rᵢ) and f(r′ᵢ) come from step 235 and g(aᵢ, tᵢ) comes from step 250, and the total healthcare cost difference (with and without transportation assistance) for patient i is

$$Δ c ( {r}_{0} , i ) = \{ \begin{matrix}[ f ( {r}_{i}^{′} ) - f ( {r}_{i} ) ] + g ( {a}_{i} , {t}_{i} ) & , if {r}_{0} < {r}_{i} \\ 0 & , if {r}_{0} \geq {r}_{i}\end{matrix} .$$

where Δc(r₀, i) is actually a modified step function. Assume the original

$$Heaviside H ( x ) = \{ \begin{matrix}0 , x < 0 \\ 1 , x > 0\end{matrix} , \\ then \\ Δ c ( {r}_{0} , i ) = H ( {r}_{i} - {r}_{0} ) \cdot \{ [ f ( {r}_{i}^{′} ) - f ( {r}_{i} ) ] + g ( {a}_{i} , {t}_{i} ) \} .$$

For all patients (total number of patients=N, scheduled at times {tᵢ}, i=[1, . . . , N]), the overall total healthcare cost difference function ΔC(r₀, {tᵢ}) after applying transportation service is

$$Δ C ( {r}_{0} , \{ {t}_{i} \} ) = {\sum }_{i = 1}^{N} Δ c ( {r}_{0} , i ) = {\sum }_{i = 1}^{N} H ( {r}_{i} - {r}_{0} ) \cdot [ f ( {r}_{i}^{′} ) - f ( {r}_{i} ) ] + g ( {a}_{i} , {t}_{i} ) , {t}_{i} ∈ {T}_{i} .$$

Then the scheduling optimization module 115 finds the optimal r₀ and list of tᵢ which produce minimum ΔC(r₀, {tᵢ}), using the overall total healthcare cost difference function ΔC(r₀, {tᵢ}), with constraints of tᵢ∈Tᵢ.

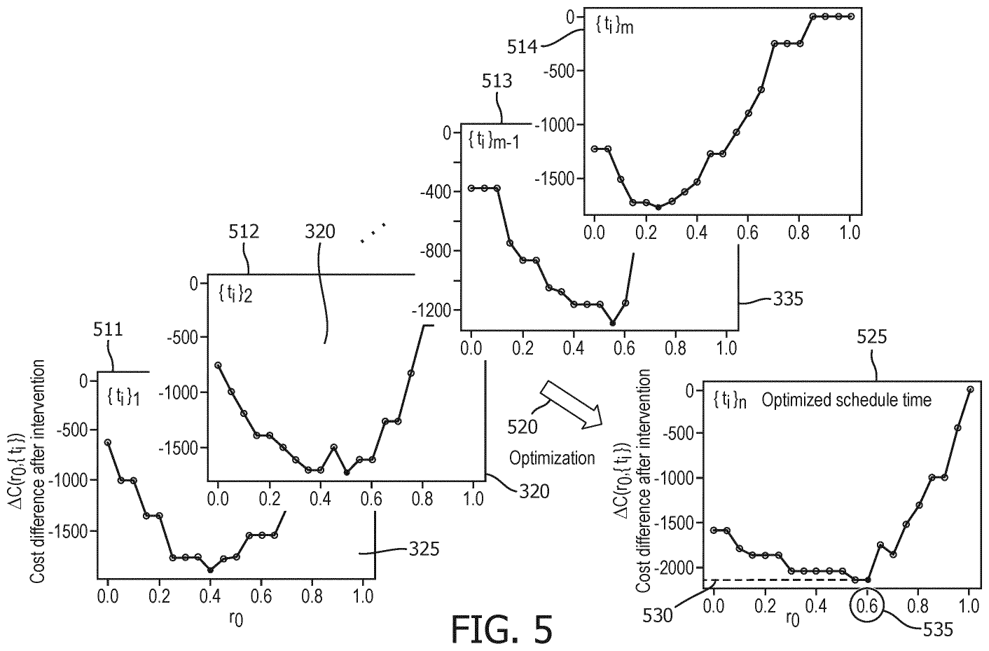

FIG. 5 illustrates an example of scheduling optimization module finding the optimal r₀ and list of tᵢ which produces a minimum overall healthcare cost difference ΔC(r₀, {tᵢ}). Plots 511, 512, 513, and 514 of ΔC(r₀, {tᵢ}) versus r₀ are shown. After optimization 520, the optimal value of r₀ 535 have the minimum overall healthcare cost difference 530 is determined. This value of r₀ may then be used to find the specific time tᵢ for each patient.

The population selection and scheduling module 120 carries out steps 260 to 270 of the method 200. The population selection and scheduling module 120 selects a patient population 260 using optimal r₀ based on step 255 and coordinates transportation service for them 265. The population selection and scheduling module 120 schedules patient i to time tᵢ (i=1:N) 270 based on step 255.

The patient scheduling system solves the technological problem of scheduling medical appointments for patients to avoid increased health care cost and poorer health outcomes due to patients missing medical appointments due to a lack of transportation. The patient scheduling system may be used to identify patients at risk of a no-show and that could benefit from transportation services and the coordination of transportation assistance. These patients would receive transportation assistance in order to make it to the medical appointment. Further, the patient scheduling system determines the best time to schedule appointments for patients in order to reduce the cost of the transportation services. An overall healthcare cost difference (with and without transportation assistance) function across a number of patients under care, may be used to perform an optimization to identify a population of patients that would benefit from transportation services and provide the greatest health savings by avoiding missed medical appointments.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for scheduling patients for medical appointments, comprising:
predicting the no-show risk and no-show cost for the patients;
forecasting the cost of a transportation assistance service for the patients;
optimizing the scheduling of patients based upon cost of the transportation assistance service, the no-show risk, and the no-show cost;
selecting a population of patients to receive the transportation assistance service; and
scheduling the population of patients for their medical appointment and transportation assistance service.

2. The method of claim 1, wherein predicting the no-show risk and no-show cost for the patients includes training no-show risk model that predicts the no-show risk.

3. The method of claim 2, wherein predicting the no-show risk for a patient includes calculating the no-show risk for the patients based upon their transportation availability and calculating the no-show risk for the patients when the transportation assistance service is provided.

4. The method of claim 3, wherein predicting the no-show risk and no-show cost for the patients includes determining a healthcare cost function based upon the no-show risk of the patients and the cost of a missed medical appointment.

5. The method of claim 4, wherein the cost of the missed medical appointment is based upon at least one of idle time of medical providers, medical equipment, medical facilities, and decreased health outcome due to the missed appointment.

6. The method of claim 1, wherein training no-show risk model that predicts the no-show risk includes using least absolute shrinkage and selection operator (LASSO) regression or random forest.

7. The method of claim 1, wherein predicting the no-show risk and no-show cost for the patients includes at least one of the following data: socioeconomic factors; income; employment; insurance coverage; age; gender; social support; vehicle availability; public transport availability; previous appointment records; electronic medical records; prior appointment attendance; and prior cost records.

8. The method of claim 1, wherein forecasting the cost of a transportation assistance service for the patients includes producing a model of the cost of the transportation assistance service based upon a patient address, a medical appointment location, and time of service.

9. The method of claim 8, wherein forecasting the cost of a transportation assistance service for the patients includes collecting available times for appointments for the patients.

10. The method of claim 9, wherein forecasting the cost of a transportation assistance service for the patients includes using the model of the cost of the transportation assistance service with available patient times as inputs.

11. The method of claim 10, wherein optimizing the scheduling of patients includes determining the total healthcare cost difference with and without transportation assistance for the patients based upon no-show cost for the patients, the cost of the transportation assistance services for the patients, and a risk threshold value.

12. The method of claim 11, wherein optimizing the scheduling of patients includes determining the risk threshold value that produces the lowest total healthcare cost difference with and without transportation assistance for the patients.

13. The method of claim 12, wherein selecting a population of patients to receive the transportation assistance service is based upon the determined risk threshold value.

14. The method of claim 8, wherein model of the cost of the transportation assistance service is one of a recurrent neural network, a long short-term memory (LSTM) recurrent neural network, a gated recurrent unit (GRU) neural network, and a time series analysis model.

15. A non-transitory machine-readable storage medium encoded with instructions for scheduling patients for medical appointments, comprising:
instructions for predicting the no-show risk and no-show cost for the patients;
instructions for forecasting the cost of a transportation assistance service for the patients;
instructions for optimizing the scheduling of patients based upon cost of the transportation assistance service, the no-show risk, and the no-show cost;
instructions for selecting a population of patients to receive the transportation assistance service; and
instructions for scheduling the population of patients for their medical appointment and transportation assistance service.

16. The non-transitory machine-readable storage medium of claim 15, wherein instructions for predicting the no-show risk and no-show cost for the patients includes instructions for training no-show risk model that predicts the no-show risk.

17. The non-transitory machine-readable storage medium of claim 16, wherein instructions for predicting the no-show risk for a patient includes instructions for calculating the no-show risk for the patients based upon their transportation availability and instructions for calculating the no-show risk for the patients when the transportation assistance service is provided.

18. The non-transitory machine-readable storage medium of claim 17, wherein instructions for predicting the no-show risk and no-show cost for the patients includes instructions for determining a healthcare cost function based upon the no-show risk of the patients and the cost of a missed medical appointment.

19. The non-transitory machine-readable storage medium of claim 18, wherein the cost of the missed medical appointment is based upon at least one of idle time of medical providers, medical equipment, medical facilities, and decreased health outcome due to the missed appointment.

20. The non-transitory machine-readable storage medium of claim 15, wherein instructions for training no-show risk model that predicts the no-show risk includes using least absolute shrinkage and selection operator (LASSO) regression or random forest.

21. The non-transitory machine-readable storage medium of claim 15, wherein instructions for predicting the no-show risk and no-show cost for the patients includes at least one of the following data: socioeconomic factors; income; employment; insurance coverage; age; gender; social support; vehicle availability; public transport availability; previous appointment records; electronic medical records; prior appointment attendance; and prior cost records.

22. The non-transitory machine-readable storage medium of claim 15, wherein instructions for forecasting the cost of a transportation assistance service for the patients includes instructions for producing a model of the cost of the transportation assistance service based upon a patient address, a medical appointment location, and time of service.

23. The non-transitory machine-readable storage medium of claim 22, wherein instructions for forecasting the cost of a transportation assistance service for the patients includes instructions for collecting available times for appointments for the patients.

24. The non-transitory machine-readable storage medium of claim 23, wherein instructions for forecasting the cost of a transportation assistance service for the patients includes using the model of the cost of the transportation assistance service with available patient times as inputs.

25. The non-transitory machine-readable storage medium of claim 24, wherein instructions for optimizing the scheduling of patients includes instructions for determining the total healthcare cost difference with and without transportation assistance for the patients based upon no-show cost for the patients, the cost of the transportation assistance services for the patients, and a risk threshold value.

26. The non-transitory machine-readable storage medium of claim 25, wherein instructions for optimizing the scheduling of patients includes instructions for determining the risk threshold value that produces the lowest total healthcare cost difference with and without transportation assistance for the patients.

27. The non-transitory machine-readable storage medium of claim 26, wherein instructions for selecting a population of patients to receive the transportation assistance service is based upon the determined risk threshold value.

28. The non-transitory machine-readable storage medium of claim 22, wherein model of the cost of the transportation assistance service is one of a recurrent neural network, a long short-term memory (LSTM) recurrent neural network, a gated recurrent unit (GRU) neural network, and a time series analysis model.
